(19) United States
(12) Patent Application Publication
(10) Pub. No.: US 2025/0278513 A1
(43) Pub. Date: Sep. 4, 2025

(54) DATA GENERATION APPARATUS AND NON-TRANSITORY COMPUTER READABLE MEDIUM

(71) Applicant: FUJIFILM Corporation, Tokyo (JP)

(72) Inventor: Takanobu OTSUBO, Tokyo (JP)

(73) Assignee: FUJIFILM Corporation, Tokyo (JP)

(21) Appl. No.: 19/064,601

(22) Filed: Feb. 26, 2025

(30) Foreign Application Priority Data
Feb. 29, 2024 (JP) 2024-029736

(51) Int. Cl. G06F 21/62; G16H 10/60

(52) U.S. Cl. CPC G06F 21/6245; G16H 10/60

(57) ABSTRACT

The data generation apparatus according to the embodiment of the present invention includes a processor, in which the processor is configured to store consented data for which consent to use data in a first application is obtained from a data provider, request the data provider for re-consent to use the consented data in a second application different from the first application, and generate re-consented data obtained by duplicating the consented data based on acquisition of the re-consent. Re-consented processed data obtained by processing the re-consented data based on the second application is provided to a data usage apparatus.

### CROSS-REFERENCE TO RELATED APPLICATIONS

This application claims priority under 35 U.S.C § 119(a) to Japanese Patent Application No. 2024-029736 filed on 29 Feb. 2024. The above application is hereby expressly incorporated by reference, in its entirety, into the present application.

### BACKGROUND OF THE INVENTION

### 1. Field of the Invention

The present invention relates to a data generation apparatus and a non-transitory computer readable medium storing a computer-executable program.

### 2. Description of the Related Art

In a medical institution that collects personal medical data through a health checkup, medical data and consent to use the medical data for diagnosis, treatment, and the like for the health of the individual are collected from a data provider who is an examinee of the health checkup, and the medical data is effectively used for an application in accordance with the consent. The personal information protection laws of each country, such as the General Data Protection Regulation (GDPR), assume that the data provided by the data provider collected by the intention of the data manager is effectively used in accordance with the content consented in advance by the data provider. In the effective usage of the collected medical data, it is possible to effectively use the medical data for a use application other than the personal health purpose, such as contribution to industry, by obtaining the consent of the data provider.

JP2006-201830A discloses that patient consent data for use for other purposes, which is different from the original use purpose, is stored for medical information used for diagnosis or treatment purposes in a medical institution, and the medical information is edited or processed based on the patient consent data. Specifically, in a case where medical information of a patient is used for clinical trials, research, education, or the like, which are purposes other than diagnosis or treatment, data processing is performed on the medical information using the patient consent data for the use for other purposes stored in advance.

### SUMMARY OF THE INVENTION

On the other hand, JP2006-201830A describes that consent to use medical information for the use for other purposes is acquired in advance and stored, but does not describe a method of using data for an application in which consent is not obtained in a case of data collection. In a case of an application other than diagnosis or treatment, since the use application and the country of transit are confirmed after the data user is decided, even in a case where consent is acquired assuming a state in which the use application is not clear, it is often not possible to respond to the newly emerged use application. Therefore, it is difficult to acquire consent for all the intended use application from the beginning and to effectively use the consent.

In the method of the related art, in order to use the medical data for newly emerged use applications (use for other purposes), it is necessary to acquire consent again from the data provider and to collect the data again. However, it is not easy to re-collect the data from the individual. In addition, a large amount of data collected in the past cannot be used and is wasted. Therefore, it is required to make it possible to use the collected personal data for an application different from the consent in a case of collection.

An object of aspects of the present invention is to provide a data generation apparatus and a non-transitory computer readable medium storing a computer-executable program that can use accumulated personal data for an application different from an application of prior consent.

A data generation apparatus according to an aspect of the present invention comprises a processor, in which the processor is configured to generate consented data for which consent to use data in a first application is obtained from a data provider, request the data provider for re-consent to use the consented data in a second application different from the first application, and generate re-consented data obtained by duplicating the consented data based on acquisition of the re-consent.

It is preferable that the processor is configured to decide a range of the consented data to be duplicated in accordance with the second application, and generate the re-consented data including a part of the consented data based on the range.

It is preferable that the processor is configured to process the re-consented data based on the second application to generate re-consented processed data.

It is preferable that the processor is configured to acquire necessary data required for use of the second application in a case where the re-consented data is insufficient for use of the second application.

It is preferable that the processor is configured to generate time-series data, which is the re-consented processed data, by combining the acquired necessary data with the re-consented data.

It is preferable that the processor is configured to determine an asset value of the consented data, the re-consented data, and the re-consented processed data, and decide a consideration to be paid to the data provider for the use of the second application based on the asset value.

It is preferable that the processor is configured to present the re-consented data and the re-consented processed data to the data provider together with the asset value.

It is preferable that the processor is configured to present the consent and the consented data to the data provider together with the request for the re-consent.

It is preferable that the processor is configured to, in a case where the second application matches the first application, use the consented data for the second application.

It is preferable that the processor is configured to process the re-consented data based on the second application for performing data analysis.

A non-transitory computer readable medium for storing a computer-executable program according to another aspect of the present invention causes a computer to realize a function of generating consented data for which consent to use data in a specific first application is obtained from a data provider, a function of requesting the data provider for re-consent to use the consented data in a second application different from the first application, and a function of generating re-consented data obtained by duplicating the consented data based on acquisition of the re-consent.

According to the aspects of the present invention, a data generation apparatus and a non-transitory computer readable medium storing a computer-executable program that can use accumulated personal data for an application different from an application of prior consent are provided.

### BRIEF DESCRIPTION OF THE DRAWINGS

FIG. 1 is a schematic diagram of a data generation system.

FIG. 2 is a block diagram showing a function of a data generation apparatus.

FIG. 3 is an explanatory diagram of a procedure performed by the data generation system before acquisition of re-consent.

FIG. 4A is an explanatory diagram of a case where only the re-consent is acquired and FIG. 4B is an explanatory diagram of a case where the re-consent and necessary data are re-collected in use of consented data in a second application that does not match a first application.

FIG. 5 is an explanatory diagram of processing of re-consented data and provision of data in a first example.

FIG. 6 is an explanatory diagram in which anonymization processing is performed on the re-consented data.

FIG. 7 is an explanatory diagram of processing of re-consented data and provision of data in a second example.

FIG. 8 is an explanatory diagram in which data addition is performed on the re-consented data.

FIG. 9 is an explanatory diagram of processing of re-consented data and provision of data in a third example.

FIG. 10 is an explanatory diagram of processing of re-consented data and provision of data in a fourth example.

FIG. 11 is a flowchart showing a series of flows in data usage in which re-consent for the second application different from the first application is acquired.

### DESCRIPTION OF THE PREFERRED EMBODIMENTS

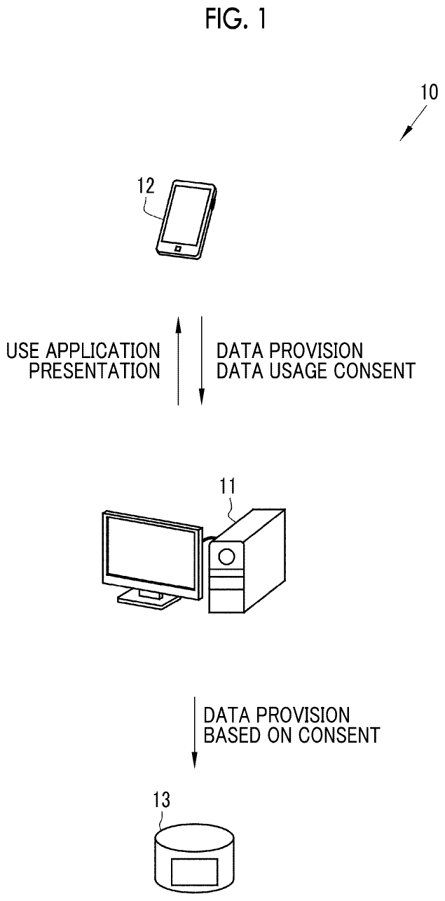

As shown in FIG. 1, a data generation system 10 includes a data generation apparatus 11 that stores acquired personal data and consent data of the personal data, a provider terminal 12 that provides the personal data to the data generation apparatus 11, and a data usage apparatus 13 that uses the data provided from the data generation apparatus 11. The data generation apparatus 11, the provider terminal 12, and the data usage apparatus 13 are managed by individuals or organizations having independent authorities.

The data generation apparatus 11 collects and stores personal data in accordance with a use application based on an instruction of a manager, and provides the personal data to the data usage apparatus 13 managed by a third party based on the consent data. The provider terminal 12 is a personal computer (PC) or a smartphone owned by the data provider that can communicate with the data generation apparatus 11, and browses the own data and the consent data, operates the data, and creates the consent data. The data usage apparatus 13 is managed by a data user, such as a corporation, an organization, or an administration, and effectively uses the personal data for which consent has been obtained.

The personal data includes personal identification information that can identify or specify the data provision. The personal identification information is information such as a name, a gender, a date of birth, a family history, a history of hospital visits, an address, an email address, a medical history, a medication history, and an ID in each data. The personal identification information refers to general information that can specify an individual by combination, such as personal image information such as a rare region included in the image, a rare numerical value or a result in each data.

In a case where the personal data is medical data, the data generation apparatus 11 is a medical institution, a personal health record (PHR) service provider, a medical device management company, or the like, and acquires the personal data from an information terminal that records an examination in the medical institution or a medical device used by an examinee (data provider). The data usage apparatus 13 is an analysis institution, a research institution, a pharmaceutical company, an insurance company, a medical device manufacturer, or the like. The medical data is height and weight data, blood pressure data, blood data, urine examination result, health checkup findings, diagnostic information, endoscopic examination result, X-ray examination result, and the like of a data provider. The endoscopic examination result and the X-ray examination result include image data. The use application of the medical data is “diagnosis”, “health analysis”, “research”, “insurance”, “pharmaceutical”, “digital marketing”, “clinical trial recruiting”, “learning data”, and the like.

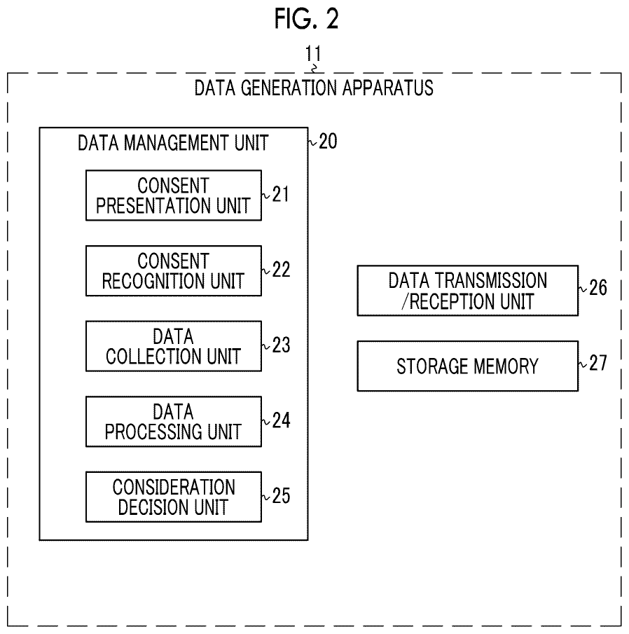

As shown in FIG. 2, the data generation apparatus 11 comprises a data management unit 20, a data transmission/reception unit 26, and a storage memory 27, and the data management unit 20 includes a consent presentation unit 21, a consent recognition unit 22, a data collection unit 23, a data processing unit 24, and a consideration decision unit 25. The data generation apparatus 11 also has a function of an input reception unit (not shown), and the input of the user who is the manager or the like of the organization to which the data generation apparatus 11 belongs can be received by an operation through a user interface (UI) such as a mouse operation or a keyboard operation. The input includes a control of processing and an instruction of individual processing for each item.

The data generation apparatus 11 is a computer, such as a personal computer or a workstation, in which an application program for realizing a predetermined function is installed. The computer comprises a central processing unit (CPU) which is a processor, a memory, a storage, and the like, and realizes various functions by a program or the like stored in the storage. Using the above functions, the data generation apparatus 11 can effectively use the collected data for an application different from the prior consent.

The data management unit 20 stores and manages the personal data collected from the data provider and the consent data associated with each personal data. In addition, the data management unit 20 decides the use application of the personal data, checks the consent data in the personal data, requests the recipient for consent, processes the data in accordance with the use application, and calculates the price of the stored medical data.

In the consent presentation unit 21, the usage permission conditions are presented to the data provider based on the data usage request from the data generation apparatus 11 or the data usage apparatus 13 or the data provision offer from the data provider, and the consent is requested. The consent is obtained in a case where the data provider accepts the usage permission conditions. The consent to be acquired may be for uncollected data collected after the consent is acquired, or may be for collected data.

The usage permission condition is decided based on the use application or output destination information for the personal data provided by the data provider. The output destination information includes information on a country or corporation to which the data usage apparatus 13 belongs. In addition, the output destination information may include the information that can determine whether or not the country or corporation has the same relationship with the output source.

The function of requesting consent by the consent presentation unit 21 may be a function of transmitting data to the provider terminal 12, to which the intention for the usage permission conditions can be input, or may be a function of creating a consent request document in which the usage permission conditions are presented to the data provider on a paper medium. In addition, in a case where the usage permission conditions are presented to the data provider and the consent is requested, a sentence or the like for fulfilling the accountability is also presented.

The consent request for the same data provider may be repeated in accordance with the addition of the use application. For example, the consent request for the second application to the data provider is made in a case where the second application different from the first application occurs for the consented data for which the consent for the first application is obtained, and in a case where a third application different from the first application and the second application occurs, the consent request for the third application is made to the data provider.

In the consent recognition unit 22, the consent to the usage permission conditions by the data provider is recognized, and consent data indicating that the usage consent is obtained is created. The created consent data is added to the corresponding personal data. In the consent data, data of the usage consent of the first application is referred to as first application consent data, data of usage consent of the second application is referred to as second application consent data, and data of the usage consent of the third application is referred to as third application consent data. Data collection, data processing, and data provision are performed based on the consent data.

The data collection unit 23 collects data based on the usage permission conditions for which consent is obtained from the data provider, and stores the data in the storage memory 27. In a case where there is insufficient use of a new application such as the second application in the accumulated data, the data collection unit 23 requests the data provider for the necessary data required for the use of the second application together with re-consent or after the re-consent is acquired, and collects the necessary data. The re-collected necessary data is stored in the storage memory 27. The data to be re-collected may be only the data of the items necessary for the new application. The necessary data is, for example, the latest personal data. Since personal data such as a body and a history of an individual becomes more inaccurate as time passes and the newer the personal data the more useful, a validity period of a creation date of raw data in the personal data is designated to be within one year or the like in the data usage request. In addition, the storage of the data includes storing the data in a separate device such as a cloud server, in addition to storing the data in the storage memory 27. The storage of the data in the separate device may be switched to the storage in the storage memory 27 by the data manager freely, or may be automatically switched by the data generation apparatus 11 based on the use application or the like.

The data processing unit 24 processes the personal data duplicated from the storage memory 27 based on the use application. The personal data is stored in the storage memory 27, and the processing is performed by duplicating the personal data (consented data) to which the first application consent data is added. The processing includes anonymization processing, accuracy deterioration processing, information addition processing, integration processing, and the like. Data obtained by performing processing suitable for the new application can be stored in the storage memory 27 and can be treated as an information asset.

In the duplication of the consented data, a range to be duplicated may be decided in accordance with the use application, and the duplication data including a part of the consented data may be acquired. In the first application, the entire consented data is used, while in the second application, the consented data may be partially used. In that case, a range of the consented data, which is stored in the storage memory 27, to be duplicated is decided based on the second application, and re-consented data including a part of the consented data is generated based on the decided range. By deciding a range to be duplicated and duplicating data to be used for a new application, it is possible to reduce the time and effort for duplicating unnecessary items in the consented data and the capacity required for storage. In addition, it is more efficient than performing partial deletion after duplication.

In the anonymization processing, the data user performs deletion of information including replacing with meaningless characters or character strings or masking of painting with black with respect to personal identification information (personal information) that can identify an individual in personal data, so that the data user cannot identify the individual who is the data provider from the personal data. Even in the image, the portion of the image to which the medical checkup ID is added is masked. Instead of replacing or deleting all the items of the personal identification information, the personal identification information may be converted into information that cannot identify the individual by the accuracy deterioration processing.

In the accuracy deterioration processing, rounding to one or two significant figures or conversion to a category indicating a position of the examination value with respect to the normal value range is performed by rounding off a numerical value of the examination value, truncating a fractional part, or the like. For example, the date of birth is converted into only the year of birth or age information such as “20 to 24 years old” or “20s”, and the address is divided into a category of local governments such as “Tokyo”. The capacity of data can be reduced and personal information can be protected by the accuracy deterioration processing.

In the information addition processing, a classification result or an analysis result obtained by classifying or analyzing information indicating checking or reliability guarantee of a data manager, a measurement value included in personal data, or the like is added. The information to be added may be created by applying data discrimination criteria or the like, which is held by the data generation apparatus 11 or acquired from the outside, to a numerical value such as a measurement value. In addition, a classification result obtained by classifying the image data by a classifier trained by machine learning may be added.

In the integration processing, a plurality of pieces of personal data are integrated and processed into one piece of data. The integration may be performed on the personal data of the same person or the personal data of different persons.

The integration of the personal data of the same person is the generation of the personal data in which the personal data having different items is collected, or the generation of the time-series data using data having the same item and different measurement dates. The integration of the personal data of different persons is the creation of comparison data in which the same items included in each personal data are extracted.

The consideration decision unit 25 decides the consideration to be paid to the data provider based on the value of the data to be provided to the data user, such as the consented data, the re-consented data, and the re-consented processed data. The value of the data is, for example, an asset value determined by a price calculation criteria decided in a data usage request or the like by the data user. Even in the case of the same data, since the value varies depending on the application, the consideration is decided for each use application.

In a case where the consideration decided by the consideration decision unit 25 is money, the consideration is a price excluding the share of the data generation apparatus 11 from the price based on the determined asset value. The share of the data generation apparatus 11 includes a cost of data storage and a cost generated by processing. In addition, in a case where the consideration is other than money, the consideration may be the result of research corresponding to the data provision, a service individualized for each data provider, or information created for the data provider.

The asset value of the data determined by the consideration decision unit 25 may be presented to the data provider through the provider terminal 12 together with the target consented data, the re-consented data, and the re-consented processed data. The data provider can manage and operate the provided data, as the information asset, for each consented use application by grasping the asset value.

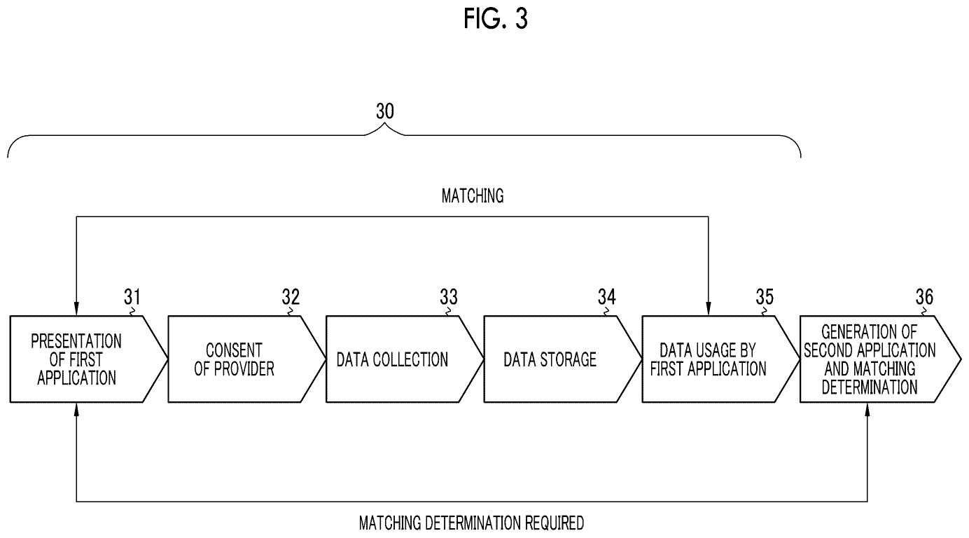

The data usage by the first application, which is executed before the data usage by the second application, will be described. As shown in FIG. 3, a pre-execution procedure 30 is executed before obtaining the consent for the second application in the data generation system 10. The pre-execution procedure 30 includes a first application presentation procedure 31, a provider consent procedure 32, a data collection procedure 33, a data storage procedure 34, and a first application usage procedure 35. After the pre-execution procedure 30 ends, a second application matching determination procedure 36 is performed by any of the data provider, the data manager, or the data user suggesting the data usage in the second application different from the first application.

In the first application presentation procedure 31, the data generation apparatus 11 presents the usage permission conditions related to the first application in which the data is used for diagnosis or treatment purposes to the data provider. In the provider consent procedure 32, the consent (permission) of the data provider to use the data for the first application by submitting a written document or inputting data is acquired. In the data collection procedure 33, the data used for the first application is collected from the provider terminal 12 or the examination device used by the data provider. In the data storage procedure 34, consented data to which the consent data is added is stored in the collected data. In the first application usage procedure 35, the stored consented data is used for the first application. The pre-execution procedure 30 may be ended by the data storage procedure 34 without performing the first application usage procedure 35.

In the first application usage procedure 35, the use application and the consent content match the use in accordance with the consent obtained in the provider consent procedure 32, but it is necessary to determine whether the second application is the use application included in the consent content. In the second application matching determination procedure 36, the matching determination as to whether or not the second application matches the consent content in which the consent to the use application of the first application is given is performed.

In the matching determination, it is determined whether or not the use application of the data and the provision destination of the data match or are included in the consent content for the data usage in the first application. In a case where it is determined that the application is “matched”, the consented data stored in the pre-execution procedure 30 is used for the second application. In a case where the application is “matched”, for example, the first application is “anti-obesity drug pharmaceutical”, the second application is “antihypertensive drug pharmaceutical”, and since the second application is an application for using blood pressure data or related information thereof in pharmaceuticals, the second application is included in the first application. In a case where it is determined that the application is “not matched”, the consent for the contents that do not match is made again (re-consent is acquired). In any matching determination result, in a case where the data to be used for the second application is insufficient, the data is re-collected. For example, even in a case where the first application and the second application match, the data is re-collected in a case where the data in a case of the consent acquisition for the first application is old and is not suitable for the use of the second application.

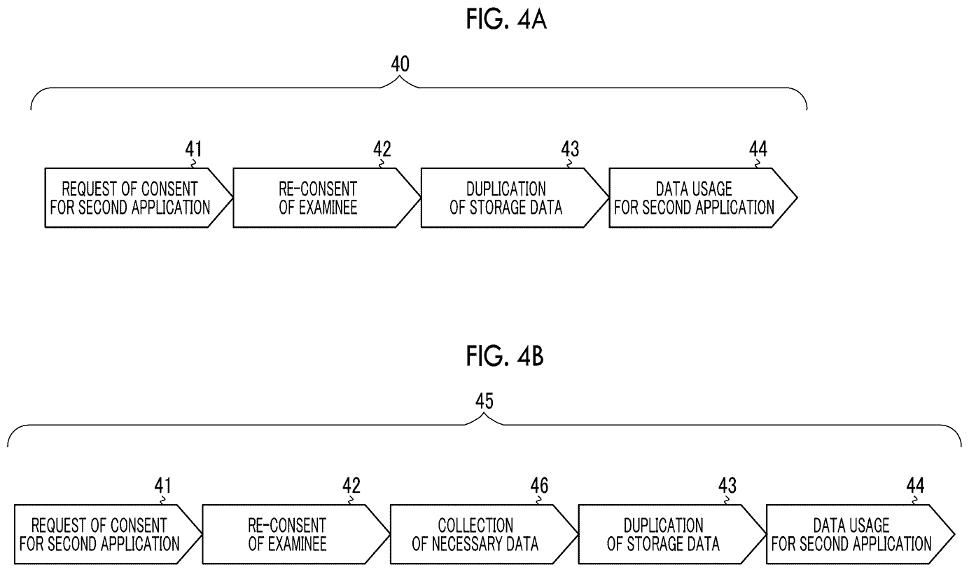

FIGS. 4A and 4B are explanatory diagrams showing a procedure of using data for which the usage consent of the first application is obtained in the second application in a case where it is determined that the application is “not matched” by the matching determination. FIG. 4A shows a re-consent acquisition procedure 40 in which there is no insufficient data and re-consent is acquired, and FIG. 4B shows a re-consent acquisition procedure 45 in which there is insufficient data and re-consent and necessary data are collected. In both the re-consent acquisition procedure 40 and the re-consent acquisition procedure 45, the consented data for which the consent to use the data for the first application is obtained from the data provider is managed by the data generation apparatus 11. The management of the data includes the holding of the data by the storage of the storage memory 27, the restriction of the data usage based on the consent, and the presentation of the data usage status to the data provider.

As shown in FIG. 4A, the re-consent acquisition procedure 40 includes a second application presentation procedure 41, a provider re-consent procedure 42, a data duplication procedure 43, and a second application usage procedure 44. In the second application presentation procedure 41, the consent presentation unit 21 presents the second application different from the first application to the data provider, and requests the consent (re-consent) to the data usage in the second application of the consented data that is stored and managed by the data generation apparatus 11. The consent in the first application and the consented data are presented to the data provider together with the request for re-consent. Accordingly, since the data provider can check the past consent contents, the use of the second application can be accurately grasped.

In the provider re-consent procedure 42, the consent recognition unit 22 receives the submission of the written document or the input of the data by the data provider to acquire the re-consent. In the data duplication procedure 43, the data processing unit 24 duplicates the stored consented data based on the acquired re-consent, and generates re-consented data to which re-consent data is added. In addition, the re-consented data may be processed based on the second application to generate the re-consented processed data. In the second application usage procedure 44, the data transmission/reception unit 26 provides the re-consented data or the re-consented processed data to the data usage apparatus 13 used for the second application. By acquiring the re-consent, the existing personal data collected in the past and stored in the data generation apparatus 11 can be used for the second application.

As shown in FIG. 4B, the re-consent acquisition procedure 45 includes a necessary data collection procedure 46 of collecting necessary data from the data provider between the provider re-consent procedure 42 and the data duplication procedure 43 in the re-consent acquisition procedure 40. In the data duplication procedure 43, the necessary data stored in the necessary data collection procedure 46 is duplicated as re-consented data together with the data accumulated for the first application. The collected necessary data may be stored as data that has been re-consented by being associated with the re-consent.

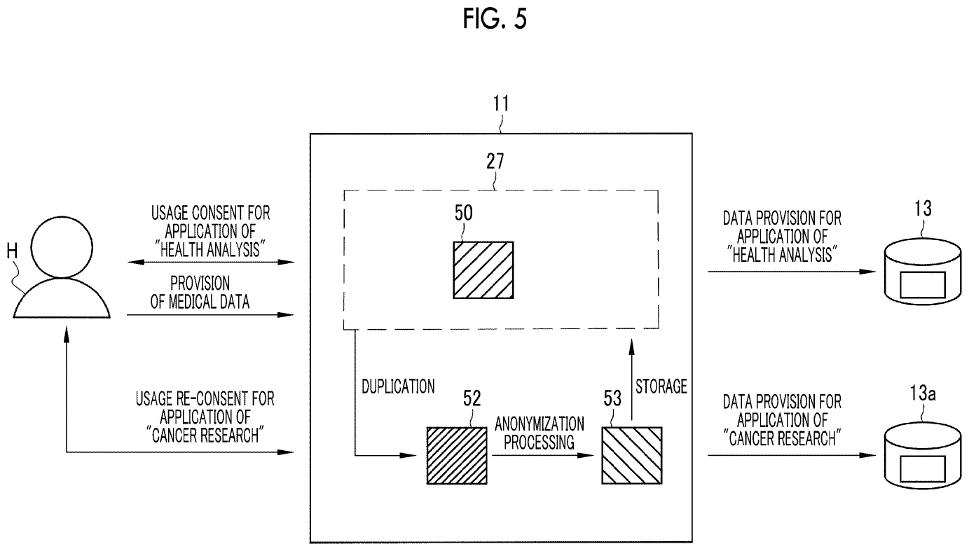

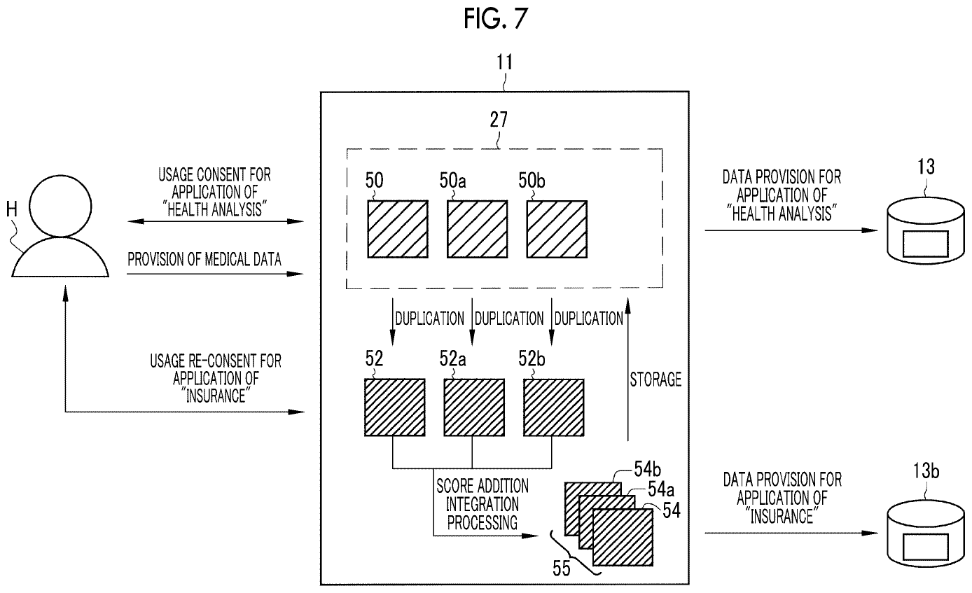

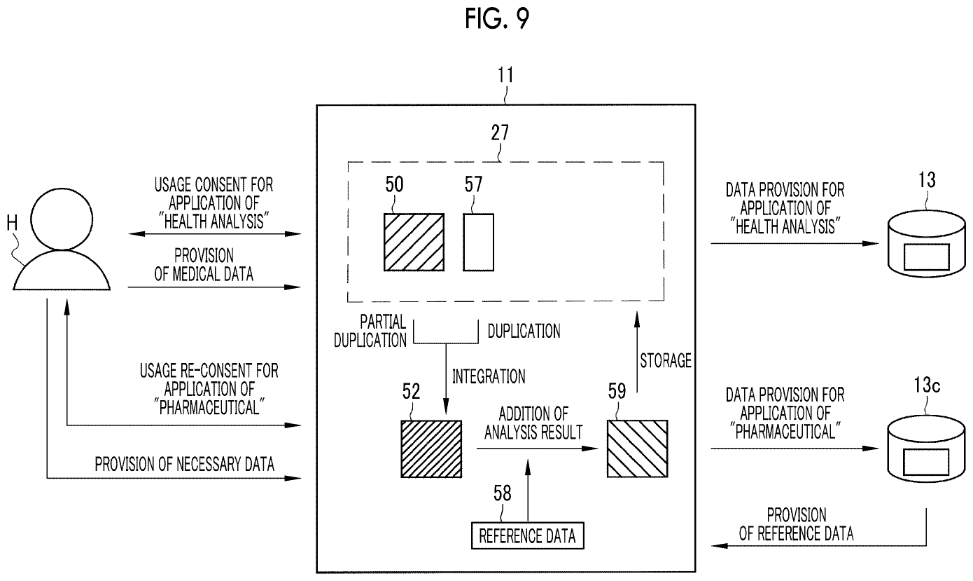

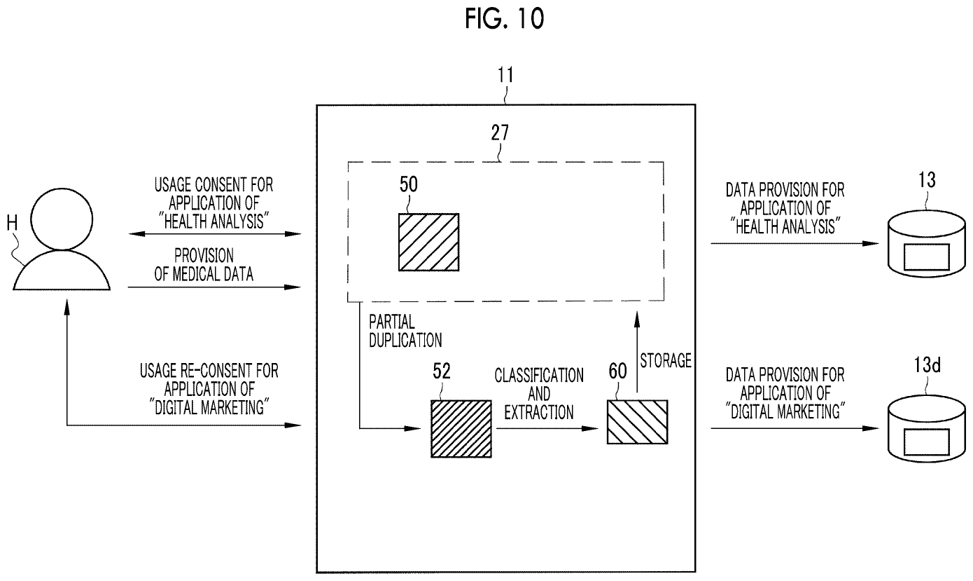

An example in which the health checkup data used for the first application of “health analysis” by the data generation apparatus 11 belonging to the health checkup center is used for the second application that does not match the first application will be described. In examples, the first example (see FIG. 5) in which the second application is “cancer research”, the second example (see FIG. 7) in which the second application is “insurance”, the third example (see FIG. 9) in which the second application is “pharmaceutical”, and the fourth example (see FIG. 10) in which the second application is “digital marketing” will be described, respectively. In any application, the data analysis is performed on the medical data by the data usage apparatus 13. In addition, the description of the contents common to the first example in the second to fourth examples will be omitted.

As shown in FIG. 5, in the use for the first application, the data provider H who is a health checkup examinee consents to the consent content (usage permission condition) for the application of “health analysis”, which is the use application for the health analysis of the data provider H, presented from the data generation apparatus 11, and provides the health checkup data to the data generation apparatus 11. The data generation apparatus 11 stores the provided health checkup data in the storage memory 27 as the consented data 50, and provides the consented data 50 to the data usage apparatus 13 belonging to an analysis institution that uses the consented data 50 for the application of “health analysis”. The data usage apparatus 13 creates a health analysis result by analyzing the consented data 50, and transmits the health analysis result to the provider terminal 12 via the data generation apparatus 11 or directly. The health checkup data is personal medical data acquired for the purpose of contributing to the health of the individual.

As shown in FIG. 5, in the first example, the data provider H re-consents to the data usage request for the data usage of the application of “cancer research”, which is the second application that does not match the first application from the data user for the consented data 50. The data generation apparatus 11 duplicates the consented data 50 by the data processing unit 24 based on the acquired re-consent (second application consent data) and creates re-consented data 52 to which the second application consent data is added. In a specific research application such as cancer research, the re-consented processed data 53 generated by processing such as anonymization processing or deletion of unnecessary information is provided to the data usage apparatus 13a belonging to the research institution. In addition, the re-consented processed data 53 is stored in the storage memory 27.

The consideration decision unit 25 calculates the price of the re-consented processed data 53 and decides the reward (consideration) to be paid to the data provider. The stored re-consented processed data 53 can be operated as data that is still valuable for the application such as “research”. In a case where the re-consented processed data 53 is provided for another application, it is possible to suppress the cost of processing.

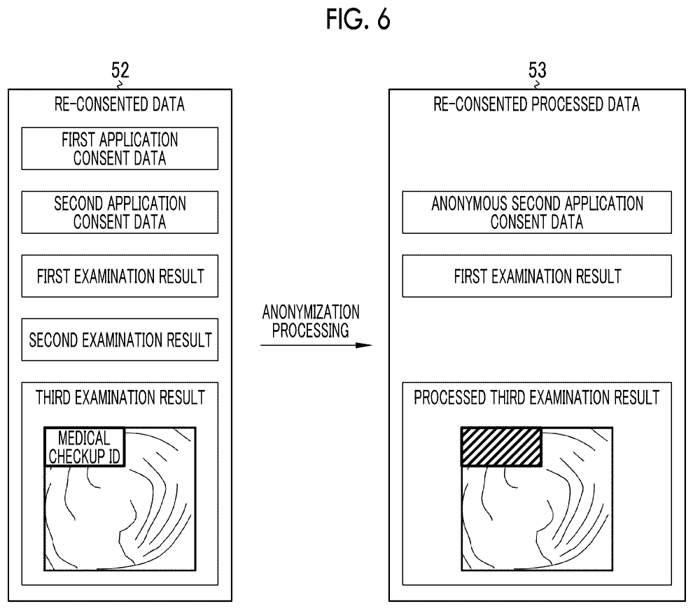

As shown in FIG. 6, the data processing unit 24 performs processing of anonymization processing or deletion of unnecessary information on the re-consented data 52 to generate re-consented processed data 53. Information that is not necessary for the use of the second application, such as the first application consent data, is deleted. The second application consent data is converted into anonymous second application consent data by performing anonymization processing on the personal identification information of the data provider.

The processing content for the examination result is decided in accordance with the examination content. For example, the examination result (first examination result) required for the second application, such as the blood examination result, is held without being processed, and the examination result (second examination result) that is not requested for the second application, such as the visual acuity examination result, is deleted. An examination result (third examination result) required for research, such as an endoscopic examination result, and that includes personal identification information of the data provider H is anonymized and converted into a processed endoscopic examination result (processed third examination result). In the generation of the re-consented data 52 by the duplication of the consented data 50, a range of data to be duplicated may be decided so that unnecessary data is not duplicated, and only the anonymization processing may be performed on the generated re-consented data 52.

As shown in FIG. 7, in the second example, the health checkup data, which is the consented data that is collected by the data generation apparatus 11 by the health checkup performed every year and is used for the first application, is used for the application of “insurance” such as the insurance product development, which is the second application. A data usage apparatus 13b belonging to the insurance company requests the usage consent of the health checkup data for three years as the use of the second application under the usage permission conditions. The data generation apparatus 11 obtains the consent (re-consent) to use the data for the application of “insurance” for the consented data 50, which is the most recent data, having a measurement date within one month, the consented data 50a having a measurement date one year ago, and the consented data 50b having a measurement date two years ago, from the data provider H. The consented data 50, 50a, and 50b are processed into the time-series data 55 having a disease risk score, which is a score indicating a disease risk, based on the second application.

The data generation apparatus 11 duplicates the consented data 50, performs score addition, which is the information addition processing, on the re-consented data 52 to which the second application consent data is added, and generates re-consented processed data 54. The re-consented data 52a is also generated for the consented data 50a, and the consented data 50a is processed into the re-consented processed data 54a. Similarly, the re-consented data 52b is generated from the consented data 50b, and the consented data 50b is processed into the re-consented processed data 54b. The re-consented data 52, 52a, and 52b are subjected to the integration processing to generate time-series data 55. The re-consented processed data 54, 54a, and 54b and the time-series data 55 are stored in the storage memory 27 as different re-consented processed data.

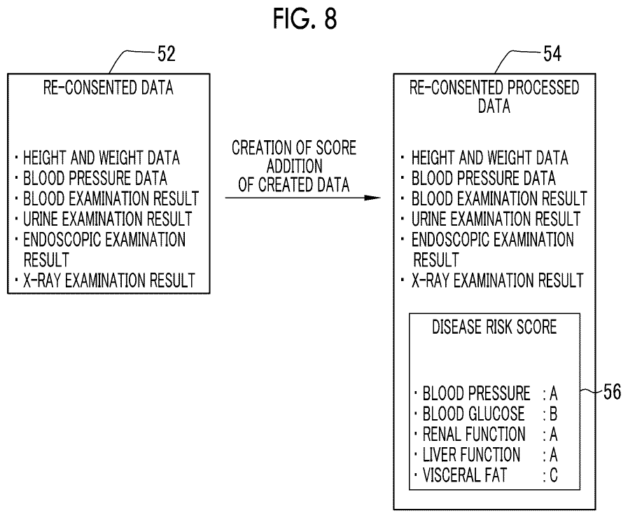

As shown in FIG. 8, in the score addition, the disease risk score 56 is created from the medical data included in the re-consented data 52 and the re-consented processed data 54 is generated by the addition. The disease risk score 56 scores the body function on a four-stage scale of A to D based on each examination result in the medical data. The score A indicates “normal”, the score B indicates “mild abnormality”, the score C indicates “observation required”, and the score D indicates “treatment required”, and the risk of the disease is low in a case of the score A or B, and the risk of the disease is high in a case of the score C or D. The score addition is similarly performed on the re-consented data 52a and 52b to acquire the re-consented processed data 54a and 54b.

The data usage apparatus 13 that uses data for the application of “insurance” can be used for calculating an insurance premium individualized for each examinee or for insurance product development, using the time-series data 55. In addition, since the time-series data 55 that can capture the change in the health state of the data provider H is valuable for the insurance industry, the data provider H can also operate the stored time-series data 55 including other insurance companies. The time-series data 55 including the diagnostic results for a plurality of years has a higher price than the total price of the individual re-consented processed data.

As shown in FIG. 9, in the third example, the consented data 50 used for the first application is used for the application of “pharmaceutical” related to the blood glucose level, which is the second application. A data usage apparatus 13c belonging to the pharmaceutical company requests the health checkup data having accurate data on the blood glucose level as the necessary item together with the re-consent. In a case where the consented data 50 does not include the accurate data of the blood glucose level, the data is re-collected from the data provider H together with the consent for the second application, and is stored in the storage memory 27 as necessary data 57. The necessary data 57 is duplicated together with the consented data 50 and is integrated into the re-consented data 52. The disease analysis is performed on the re-consented data 52 using reference data 58, and the re-consented processed data 59 to which the analysis result is added is provided to the data usage apparatus 13c.

In the disease analysis, a disease that is designated in advance or a rare disease having a morbidity rate of less than 0.1% for the population is calculated as a specific disease, and disease possibility information indicating the morbidity possibility of the data provider H is calculated as an analysis result. The reference data 58 is medical data of a specific disease patient aggregated from other medical institutions provided via the data usage apparatus 13c. In the duplication of the consented data 50, the blood data and the information on the age and the gender are selected as the range to be duplicated as useful data for “pharmaceutical” related to the blood glucose level.

In the application of “pharmaceutical”, the medical data having a morbidity possibility of a specific disease is data having a higher value than data not having a morbidity possibility of a specific disease. Therefore, the consideration decision unit 25 calculates a price in accordance with the morbidity possibility of the specific disease. The price may be decided in advance for each specific disease. In addition, in a case where the price cannot be set by the data generation apparatus 11, such as a rare disease, the price may be decided based on a suggested price of a pharmaceutical company. In a case where the data provider H is a patient of or having a high morbidity possibility of a specific disease, obtaining a new drug corresponding to the specific disease is also a consideration. In addition to obtaining a new drug, information such as new drug development information and suggestion for clinical trial recruiting is also a consideration.

As shown in FIG. 10, in the fourth example, in a case where the data provider H consents to the data usage for the consented data 50 for the application of “digital marketing”, which is the second application, the data generation apparatus 11 classifies the re-consented data 52 obtained by adding the second application consent data to the partially duplicated consented data 50, and generates re-consented processed data 60 based on the classification result. The generated re-consented processed data 60 is provided to a data usage apparatus 13d belonging to the medical device manufacturer.

In a case where the second application is an application such as “digital marketing” for market research or service conception and a specific examination value is used and an accurate examination value is not required, the range is decided to be a range in which only the age, the gender, the residence information, and the specific examination result from the consented data 50 are duplicated, and the re-consented data 52 in which a part is duplicated is divided into categories by the classification processing. For example, it is sufficient that a health state of whether an examination value measured in a health checkup falls within a normal value range or is higher or lower than the normal value range can be discriminated, and the examination value is rounded by the accuracy deterioration processing even in a case where the examination value is left.

In the classification processing, for example, the type of hypertension (such as grade I hypertension, grade II hypertension, and grade III hypertension) is classified from the blood pressure data included in the re-consented data 52, and the classification result is acquired. The re-consented processed data 60 in which the classification result is replaced with the blood pressure data of the re-consented data 52 is generated.

The re-consented processed data 54 represented by the classification result without including the detailed examination value can be operated as data that is valuable for commercial use in addition to medical device-related companies such as medical device manufacturers. For example, the data can be provided in response to the demand from the data users in a large number of commercial use, such as sports-related companies such as fitness gyms and food-related companies such as health food manufacturers. The consideration decision unit 25 can obtain a consideration for the supply of the individualized service, the request for further data provision based on the classification result, the provision of the marketing information, and the like in accordance with the provided data such as the medical device sales information.

By generating and operating the consented data, the re-consented data, or the re-consented processed data that is valuable for various applications or data users and can be provided, each data or data group can be converted into an asset.

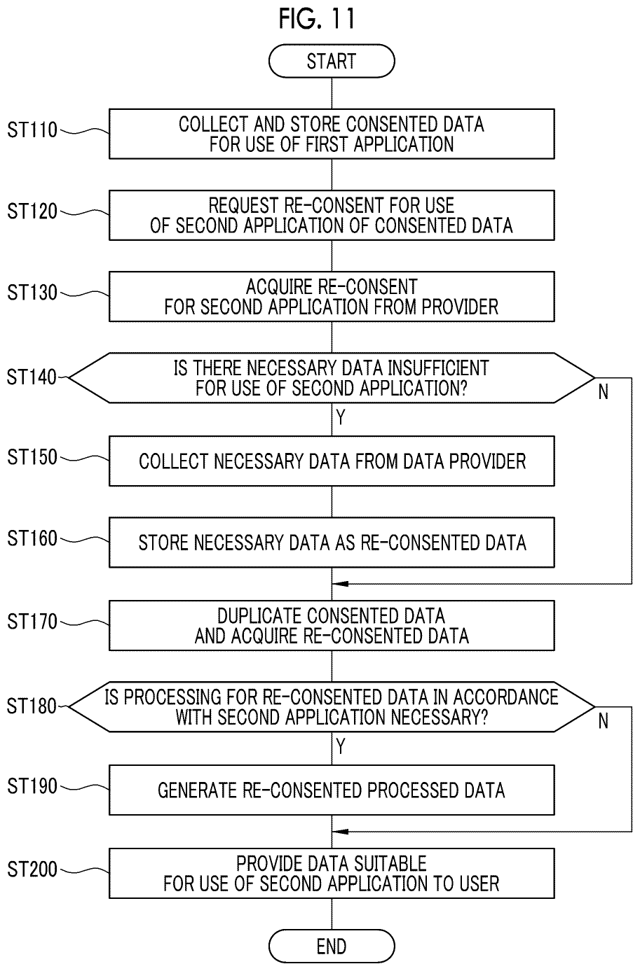

A series of flows of operations in the use of the data for which the re-consent for the second application different from the first application of the data generation system 10 according to the present embodiment is acquired will be described with reference to the flowchart shown in FIG. 11. The data generation apparatus 11 collects the consented data 50 for which the consent to the data usage in the first application has been obtained from the data provider H, and stores and manages the consented data 50 in the storage memory 27 (step ST110). In a case where the use of the consented data 50 in the second application different from the first application is suggested from the data provider H, the data generation apparatus 11, or the data usage apparatus 13, the data generation apparatus 11 presents the data provider H with the usage permission condition for the use of the second application of the consented data 50 and requests re-consent (step ST120). The data generation apparatus 11 acquires the re-consent for the use of the second application from the data provider H (step ST130).

In a case where the consented data 50 is insufficient for the use of the second application (Y in step ST140), the necessary data required for the use of the second application is acquired from the data provider H (step ST150). The acquired necessary data is stored in the storage memory 27 as the re-consented data 52a having the re-consent data (step ST160). In a case where the consented data 50 is not insufficient for the use of the second application (N in step ST140), the data acquisition is not performed.

The consented data 50 used for the second application accumulated in the storage memory 27 is duplicated, and the re-consented data 52 having the re-consent data is acquired (step ST170). In the use of the second application, in a case where the processing is necessary for the re-consented data 52 or 52a (Y in step ST180), the processing is performed based on the second application to generate the re-consented processed data 54 (step ST190). In a case where the re-consented data 52 and 52a is not required to be processed for the use of the second application (N in step ST180), the processing is not performed.

The data suitable for the use of the second application among the re-consented data 52, the re-consented data 52a, and the re-consented processed data 54 generated by the data management unit 20 is provided to the data user (step ST200). The data usage apparatus 13 uses the data provided for the second application based on the provision of the data that has been re-consented by the data generation apparatus 11.

As described above, the accumulated data can be used for the application (second application) different from the application (first application) for which prior consent is obtained. In a case where the data generation apparatus 11 can use the data for the first application or the second application (including the functions and roles of the data usage apparatus 13), the data generation system 10 may be realized by the provider terminal 12 and the data generation apparatus 11.

A modification example of the present embodiment will be described. In the above description, the form in which the data generation apparatus 11 is provided at one base in the data generation system 10 has been described, but in a case of treating a huge number of pieces of personal data, a form in which the data generation apparatus 11 is provided at each of a plurality of bases may be used. In that case, the blockchain network is realized, and each of the personal data is distributed and managed at a plurality of bases. In a case where the processing is performed, the personal data to be distributed and managed is aggregated in the data generation apparatus 11 of any base.

The data generation system 10 according to the embodiment of the present invention can also be performed on personal data that is not medical data. For example, instead of the medical data, the personal data related to the facility use information, the career information, and the electronic money use history may be used. In that case, the data usage apparatus 13 is used for creating statistics of each data, analysis, and the like.

In the above-described embodiment, the hardware structures of processing units that execute various types of processing, such as a central control unit (not shown), an input reception unit (not shown), the data management unit 20, the data transmission/reception unit 26, and the storage memory 27, are various processors as described below. The various processors include a central processing unit (CPU) that is a general-purpose processor which functions as various processing units by executing software (programs), a programmable logic device (PLD) that is a processor of which a circuit configuration can be changed after manufacturing, such as a field programmable gate array (FPGA), a dedicated electrical circuit that is a processor having a circuit configuration exclusively designed to execute various types of processing, and the like.

One processing unit may be configured by one of various processors, or may be configured by a combination of two or more processors of the same or different kinds (for example, a combination of a plurality of FPGAs or a combination of a CPU and an FPGA). In addition, a plurality of processing units may be configured by one processor. As an example in which the plurality of processing units are configured by one processor, first, there is a form in which one processor is configured by a combination of one or more CPUs and software and the processor functions as the plurality of processing units, as represented by a computer of a client or a server. Second, there is a form in which a processor that realizes functions of the entire system including the plurality of processing units with one integrated circuit (IC) chip is used, as represented by a system on chip (SoC). As described above, various processing units are configured using one or more of the various processors as a hardware structure.

Further, the hardware structures of the various types of processors are, more specifically, an electrical circuit (circuitry) in a form in which circuit elements such as semiconductor elements are combined. In addition, a hardware structure of a storage unit is a storage device such as a hard disc drive (HDD) and a solid state drive (SSD).

In addition, from the above description, the data generation apparatus described according to the following Supplementary Notes 1 to 10 can be grasped.

### Supplementary Note 1

A data generation apparatus comprising:
a processor, in which
the processor is configured to:
   store consented data for which consent to use data in a first application is obtained from a data provider;
   request the data provider for re-consent to use the consented data in a second application different from the first application; and
   generate re-consented data obtained by duplicating the consented data based on acquisition of the re-consent.

### Supplementary Note 2

The data generation apparatus according to Supplementary Note 1, in which
the processor is configured to:
   decide a range of the consented data to be duplicated in accordance with the second application; and
   generate the re-consented data including a part of the consented data based on the range.

### Supplementary Note 3

The data generation apparatus according to Supplementary Note 1 or 2, in which
the processor is configured to process the re-consented data based on the second application to generate re-consented processed data.

### Supplementary Note 4

The data generation apparatus according to Supplementary Note 3, in which
the processor is configured to acquire necessary data required for use of the second application in a case where the re-consented data is insufficient for use of the second application.

### Supplementary Note 5

The data generation apparatus according to Supplementary Note 4, in which
the processor is configured to generate time-series data, which is the re-consented processed data, by combining the acquired necessary data with the re-consented data.

### Supplementary Note 6

The data generation apparatus according to any one of Supplementary Notes 3 to 5, in which
the processor is configured to:
   determine an asset value of the consented data, the re-consented data, and the re-consented processed data; and
   decide a consideration to be paid to the data provider for the use of the second application based on the asset value.

### Supplementary Note 7

The data generation apparatus according to Supplementary Note 6, in which
the processor is configured to present the re-consented data and the re-consented processed data to the data provider together with the asset value.

### Supplementary Note 8

The data generation apparatus according to any one of Supplementary Notes 1 to 7, in which
the processor is configured to present the consent and the consented data to the data provider together with the request for the re-consent.

### Supplementary Note 9

The data generation apparatus according to any one of Supplementary Notes 1 to 8, in which
the processor is configured to, in a case where the second application matches the first application, use the consented data for the second application.

### Supplementary Note 10

The data generation apparatus according to any one of Supplementary Notes 3 to 9, in which
the processor is configured to process the re-consented data based on the second application for performing data analysis.

### Explanation of References

10: data generation system
11: data generation apparatus
12: provider terminal
13: data usage apparatus
13a: data usage apparatus
13b: data usage apparatus
13c: data usage apparatus
13d: data usage apparatus
20: data management unit
21: consent presentation unit
22: consent recognition unit
23: data collection unit
24: data processing unit
25: consideration decision unit
26: data transmission/reception unit
27: storage memory
30: pre-execution procedure
31 first application presentation procedure
32: provider consent procedure
33: data collection procedure
34: data storage procedure
35: first application usage procedure
36: second application matching determination procedure
40: re-consent acquisition procedure
41: second application presentation procedure
42: provider re-consent procedure
43: data duplication procedure
44: second application usage procedure
45: re-consent acquisition procedure
46: necessary data collection procedure
50: consented data
52: re-consented data
52a: re-consented data
52b: re-consented data
53: re-consented processed data
54: re-consented processed data
54a: re-consented processed data
54b: re-consented processed data
55: time-series data
56: disease risk score
57: necessary data
58: reference data
59: re-consented data
60: re-consented processed data
H: data provider
ST110 to ST200: step

What is claimed is:

1. A data generation apparatus comprising:
a processor, wherein
the processor is configured to:
store consented data for which consent to use data in a first application is obtained from a data provider;
request the data provider for re-consent to use the consented data in a second application different from the first application; and
generate re-consented data obtained by duplicating the consented data based on acquisition of the re-consent.

2. The data generation apparatus according to claim 1, wherein
the processor is configured to:
decide a range of the consented data to be duplicated in accordance with the second application; and
generate the re-consented data including a part of the consented data based on the range.

3. The data generation apparatus according to claim 1, wherein
the processor is configured to process the re-consented data based on the second application to generate re-consented processed data.

4. The data generation apparatus according to claim 3, wherein
the processor is configured to acquire necessary data required for use of the second application in a case where the re-consented data is insufficient for use of the second application.

5. The data generation apparatus according to claim 4, wherein
the processor is configured to generate time-series data, which is the re-consented processed data, by combining the acquired necessary data with the re-consented data.

6. The data generation apparatus according to claim 2, wherein
the processor is configured to:
determine an asset value of the consented data, the re-consented data, and the re-consented processed data; and
decide a consideration to be paid to the data provider for the use of the second application based on the asset value.

7. The data generation apparatus according to claim 6, wherein
the processor is configured to present the re-consented data and the re-consented processed data to the data provider together with the asset value.

8. The data generation apparatus according to claim 1, wherein
the processor is configured to present the consent and the consented data to the data provider together with the request for the re-consent.

9. The data generation apparatus according to claim 1, wherein
the processor is configured to, in a case where the second application matches the first application, use the consented data for the second application.

10. The data generation apparatus according to claim 2, wherein
the processor is configured to process the re-consented data based on the second application for performing data analysis.

11. A non-transitory computer readable medium for storing a computer-executable program, the computer-executable program causing a computer to realize:
a function of storing consented data for which consent to use data in a specific first application is obtained from a data provider;
a function of requesting the data provider for re-consent to use the consented data in a second application different from the first application; and
a function of generating re-consented data obtained by duplicating the consented data based on acquisition of the re-consent.
